(19) United States
(12) Patent Application Publication
(10) Pub. No.: US 2015/0250984 A1
(43) Pub. Date: Sep. 10, 2015

(54) MEDICAL LINE DEVICE AND SYSTEM

(71) Applicant: TCB-ARROW LIMITED, Camberley, Surrey (GB)

(72) Inventors: Murray Humphries, Camberley (GB); Stephen Cooper, Camberley (GB)

(21) Appl. No.: 14/432,425

(22) PCT Filed: Sep. 30, 2013
(86) PCT No.: PCT/GB2013/000413

§ 371 Date: Mar. 30, 2015

(30) Foreign Application Priority Data
Sep. 28, 2012 (GB) 1217458.7

(51) Int. Cl. A61M 25/02

(52) U.S. Cl. CPC A61M 25/02; A61M 2025/0253; A61M 2207/00

(57) ABSTRACT

The present invention relates to medical lines for the percutaneous provision of fluids into human and animal bodies. In particular, the present invention relates to a device (20) and system (10) for orienting a medical line with respect to a patient. The medical treatment of patients commonly involves the use of percutaneously inserted lines to direct fluids directly into the bloodstream, a specific organ or an internal location within the patient, or to monitor vital functions of the patient. For instance, short, peripherally-inserted, intra-arteriovenous catheters are commonly used to provide medication and direct fluids directly into the bloodstream of the patient, otherwise commonly referred to as intravenous (IV) catheters. It is common in the treatment of patients to utilize the catheter in a variety of ways. For example, a directed fluid catheter typically drains from a container positioned above the patient to feed under gravity or is delivered via an infusion pump. The fluid flows through tubing and thence into an indwelling catheter. A convenient angle for insertion of a cannula may realise an awkward angle for the medical line attached to the cannula. The present invention provides a device and system to overcome this problem.

### FIELD OF THE INVENTION

The present invention relates to medical lines for the percutaneous provision of fluids into human and animal bodies. In particular, the present invention relates to a device and system for orienting a medical line with respect to a patient.

### BACKGROUND TO THE INVENTION

The medical treatment of patients commonly involves the use of so percutaneously inserted lines to direct fluids directly into the bloodstream, a specific organ or an internal location within the patient, or to monitor vital functions of the patient. For instance, short, peripherally-inserted, intra-arteriovenous catheters are commonly used to provide medication and direct fluids directly into the bloodstream of the patient, otherwise commonly referred to as intravenous (IV) catheters. It is common in the treatment of patients to utilize the catheter in a variety of ways. For example, a directed fluid catheter typically drains from a container positioned above the patient to feed under gravity or is delivered via an infusion pump. The fluid flows through tubing and thence into an indwelling catheter. The catheter and the fluid tubing are commonly removably attached to each other by a conventional luer-lock connector. Intravenous therapy or IV therapy is the infusion of liquid substances directly into a vein. The word intravenous simply means “within a vein”. It is commonly referred to as a drip because many systems of administration employ a drip chamber, which prevents air from entering the blood stream (air embolism), and allows an estimation of flow rate.

The use of a peripheral cannula is the most common intravenous access method in both hospitals and pre-hospital services. A peripheral IV line (PVC or PIV) consists of a short catheter (a few centimeters long) inserted through the skin into a peripheral vein (any vein not inside the chest or abdomen). This is usually in the form of a cannula-over-needle device, in which a flexible plastic cannula comes mounted on a metal trocar. Once the tip of the needle and cannula are located in the vein the trocar is withdrawn and discarded and the cannula advanced inside the vein to the appropriate position and secured. Blood may be drawn at the time of insertion.

Any accessible vein can be used although arm and hand veins are used most commonly, with leg and foot veins used to a much lesser extent. In infants the scalp veins are sometimes used.

The calibre of cannula is commonly indicated in gauge, with 14 being a very large cannula (used in resuscitation settings) and 24-26 the smallest. The most common sizes are 16-gauge (midsize line used for blood donation and transfusion), 18- and 20-gauge (all-purpose line for infusions and blood draws), and 22-gauge (all-purpose pediatric line). 12- and 14-gauge peripheral lines are capable of delivering large volumes of fluid extremely fast accounting for their popularity in emergency medicine. These lines are frequently called “large bores” or “trauma lines”.

A health care professional commonly employs adhesive bandages, plasters or surgical tape to attach a catheter or medical line to a patient, to maintain the catheter or medical line in place with respect to the skin of the patient. To adjust the position of the device the adhesive bandage would have to be removed and replaced. This often leads to poor positioning of the tube because any adjustment causes unnecessary inconvenience and discomfort to the patient and wastes bandages.

It is common for the medical line to be caused to pass along a specific part of the body and may require the line to be reversed in direction or to traverse a right angle change in direction. Equally a second section of a line may need to be positioned in a specific orientation relative to a first section of the same line. As will be appreciated, when a line is inserted for addition or removal of fluids, the kinking of a line can have disastrous results: The medical treatment is not provided or, at best, is discontinuous, the removal of fluid is not performed or not performed at a sufficient rate, or monitoring or diagnosis is incomplete.

It is also to be noted that in any catheterization process, relatively frequent disconnection between the catheter and the fluid supply tube, is often required as well as dressing changes. For instance, intravenous catheterization is frequently maintained for several days, depending upon the condition of the patient. The tubing is generally replaced every 48 to 72 hours in order to maintain the sterility of the fluid and the free-flow of the fluid through the tubing. A health care professional must therefore frequently change the tubing and newly configure replacement tubing, all in a clean environment.

The health care professional can spend a great deal of valuable time applying, removing and reapplying medical lines, ensuring that kinks and the like do not occur. The frequent application and removal of the tape also commonly excoriates the patient's skin about the insertion site. Many patients also do not rest comfortably and worry about catheter dislodgement when they move, when only tape and a dressing secure the catheter in place. Taped intravenous catheters are also easily pulled out during a “routine” dressing change, especially by inexperienced health care providers. Whilst pressure sensitive adhesives can assist the healthcare professional, this can exacerbate the situation since the adhesive is placed upon the skin and removed frequently, oftentimes lifting hairs from their follicles, possibly giving rise to a comedo.

Medical tape has long been the most widely used product for securing medical devices. Paper tape for medical applications was introduced more than 50 years ago, and tape varieties made of fabric, silk, plastic and other materials have since entered the market. Standard medical tape generally exhibits low tensile strength so it can be detached or divided as needed. Although versatile and relatively inexpensive, standard tape in roll form has its drawbacks. In veterinarian usage, the animal patient would also benefit from shaving, where there site of insertion is fur/hair covered.

One drawback is contamination. Multiple studies have shown that roll tape can become contaminated with bacteria during normal clinical use, raising concerns about the potential spread of infection. In one study (D. A. Redelmeier, MD, and N. J. Livesly, MD, “Adhesive Tape and Intravascular-Catheter-Associated Infections,” JGIM, 14 Jun. 1999), researchers found that 74% of tape specimens collected from one hospital were colonised by pathogenic bacteria. In 41% of the samples, the bacterial colonies were too numerous to count. Clinicians commonly place rolls of tape in their pockets and carry them between patient sites. Portions of tape are sometimes adhered to furniture associated with the site of administration, such as bedside tables specifically, Health care providers often tear off small strips of tape and place them on the hand rail on non-sterile surfaces such as the patient's bed frame or tray tables. Clostridium and other bacteria, however, commonly exist on these surfaces and can be transferred to the patient's skin in the proximity of the insertion site. Furthermore, the area around the tape is a trap for contaminant build-up, harbouring bacteria. In addition, tape securement requires the nurse to handle the tape while wearing protective latex gloves. Tearing adhesive tape tends to produce microscopic and/or visible holes in the gloves and thus destroys glove barrier protection. Prior securement methods also have not served the patient well. It is to be noted that the use of medical lines is not restricted to the provision of IV therapy to human beings; IV therapy is commonly employed for agricultural animals such as cattle, horses and in respect of cats and dogs, in particular, where the problems of line retention are magnified since the animal patient will seek to remove any interfering forms of treatment.

Presently there are no systems that address the need for a medical line to follow a particular path about the body.

### OBJECT TO THE INVENTION

The present invention seeks to provide an improved line securement device and system. In particular, the present invention seeks to provide a medical line placement device which can adapt to various configurations of patient and positions of placement thereabout and can simply enable adjustment of position of the medical line.

Specifically, the present invention seeks to provide a simple device to enable appropriate securement of a line without problems such as crimping or blockage arising.

### SUMMARY OF THE INVENTION

In accordance with a first aspect of the present invention, there is provided a medical line retainer device comprising a first cuff, a second cuff and an arcuate channel member, the arcuate channel subtending an angular spread, the first and second cuffs being associated with first and second ends of the arcuate channel; wherein the first cuff is shaped to retain a medical line when placed therebetween, the medical line being connectable with respect to one of a patient or medical supply; wherein the second cuff is shaped to retain a second portion of said medical line, with the portion of medical line between the cuffs lying within the arcuate channel, the medical line being connectable with respect to one of a medical supply or patient; whereby the medical line at the second cuff is directed in a different direction, as determined by the angular spread of the arcuate channel.

Conveniently, the angular spread of the arcuate channel between 45° and 180°, whereby to adjust the axial direction of the medical line from 45° to 180°. This enables a medical line to be controllably directed about a person receiving medical intravenous treatment so that the path of the line is less exposed to danger and can be worn more comfortably, or in a more accessible fashion for healthcare professionals.

Many variation of the cuff are envisaged: The first cuff can comprises a closed aperture; in the alternative, it can comprise a generally ring-like member, wherein a slit defined in the ring, angled with respect to the circumference and the arcuate channel whereby a medical line can be placed through the slit and twisted such that it can lie coaxial with the channel and be resiliently retained within the cuff. As a further alternative, the first cuff could comprise an open yoke, conveniently with a gap as defined by opposing fingers of the yoke being of a width less than a width of the medical line.

Equally, the second cuff can comprise a closed aperture. Conveniently, the second cuff can comprise an open yoke or can comprise a generally ring-like member, wherein a slit defined in the ring, angled with respect to the circumference and the arcuate channel whereby a medical line can be placed through the slit and twisted such that it can lie coaxial with the channel and be resiliently retained within the cuff.

The body of the arcuate channel can have a textured surface to provide grip. This can assist a healthcare professional in the placement of a medical line.

The body of the support member can extend in a plane such that it is generally coplanar with an exterior edge of the arcuate channel. Employing a medical grade adhesive, conveniently with a foam backing, an adhesive patch can be employed to enable the device to operably adhere to a patient.

In accordance with another aspect of the invention, there is provided an intravenous medical line comprising a medical line, a cannula, a drip chamber, a fluid supply, and a medical line device comprising a first cuff, a second cuff and an arcuate channel member, the arcuate channel subtending an angular spread, the first and second cuffs being associated with first and second ends of the arcuate channel; wherein the first cuff is shaped to retain a medical line when placed therebetween, the medical line being connectable with respect to one of a patient or medical supply; wherein the second cuff is shaped to retain a second portion of said medical line, with the portion of medical line between the cuffs lying within the arcuate channel, the medical line being connectable with respect to one of a medical supply or patient; whereby the medical line at the second cuff is directed in a different direction, as determined by the angular spread of the arcuate channel.

In accordance with another aspect of the present invention, there is provided a method of preparing a medical line retainer device, said retainer device comprising a first cuff, a second cuff and an arcuate channel member, the arcuate channel subtending an angular spread, the first and second cuffs being associated with first and second ends of the arcuate channel; the method comprising the step of placing a medical line with respect to a first cuff of the retainer device, so as to retain the medical line therewith, placing the medical line with respect to a second cuff of the retainer device so, as to retain the medical line therewith, adjusting the medical line with respect to said first and second cuffs; the medical line being connectable and disconnectable with respect to one of an intravenous connection associated with a patient and a medical supply; whereby the medical line at the second cuff is directed in a different direction with respect to the first cuff, as determined by the angular spread of the arcuate channel. With respect to a patient, this enables a medical line to be controllably directed about a patient receiving medical intravenous treatment so that the path of the line is less exposed to danger and can be worn more comfortably, or in a more accessible fashion for healthcare professionals. Conveniently, the retention of the medical line with respect to each of the cuffs is one of a sliding fit. Once secured, the device stabilizes a catheter or medical device and substantially prevents catheter movement and migration. Furthermore, the present invention has at least a reduced need for the use of additional securement means such as tapes which, because of the exposed adhesive that will become a site for bacterial growth, the minimization of such exposure substantially reduces the risk of an insertion site infection.

Further aspects, features, and advantages of the present invention will become apparent from the detailed descriptions of the preferred embodiments that follow.

### BRIEF DESCRIPTION OF THE FIGURES

Some preferred embodiments of the invention will now be described, by way of example, with reference to the accompanying drawings, of which:

FIG. 1 is a view of a hand with an intravenous medical line secured thereto;

FIG. 2 is a first perspective view of a first embodiment of a device made in accordance with the present invention;

FIGS. 3-4c show the first embodiment in second, third and fourth perspective views;

FIGS. 5a-e show plan views of the first embodiment;

FIGS. 6 & 7 show an intravenous system in accordance with the present invention;

FIGS. 8a & 8b show the a second variation of the first cuff; and,

FIG. 9 shows a still further embodiment of the invention.

### DETAILED DESCRIPTION OF THE PREFERRED EMBODIMENTS

There will now be described, by way of example only, the best mode contemplated by the inventor for carrying out the present invention. In the following description, numerous specific details are set out in order to provide a complete understanding to the present invention. It will be apparent to those skilled in the art, that the present invention may be put into practice with variations of the specific.

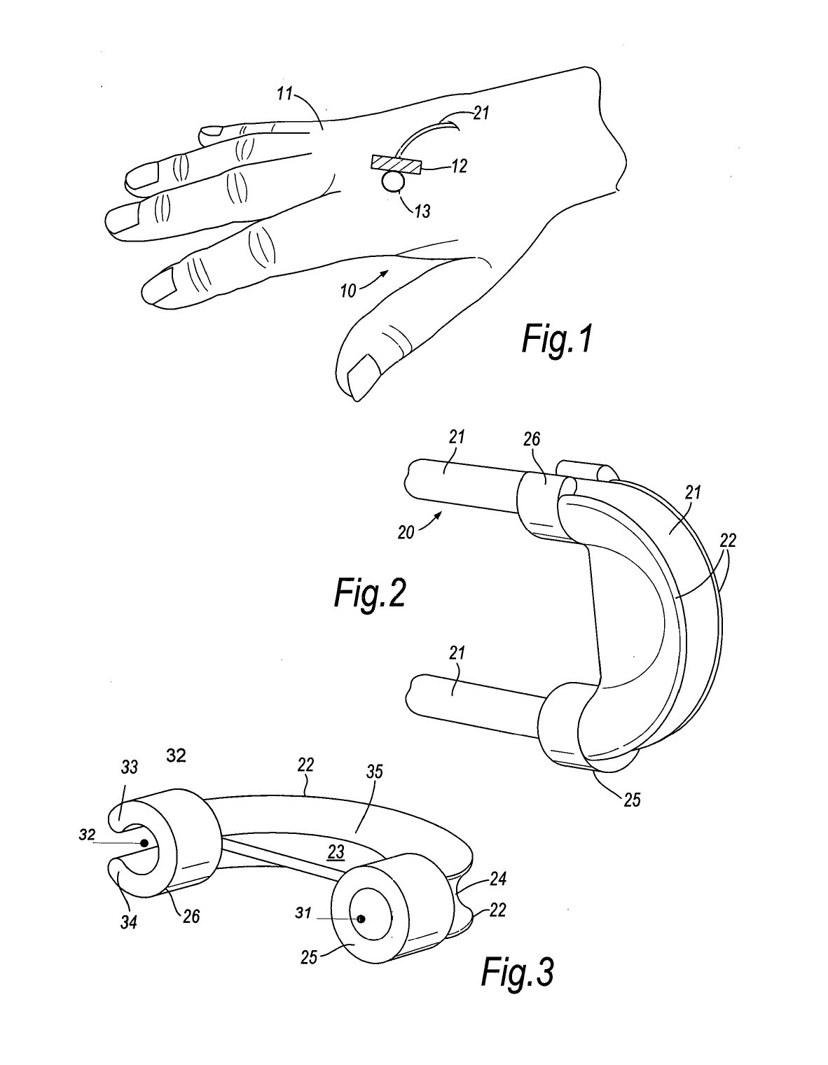

FIG. 1 shows a known medical device securement system 10 applied to a hand 11. A luer-lock and stop-cock 12 arrangement are shown coupled to an intravenous line 21, but are not coupled to any further line. As will be appreciated, although a further line is convenient to attach, it is not necessarily convenient for the patient, who may well need to be attached to a supply/drain catheter for hours at a time. In the case of an attachment being present for several tens of minutes or longer, the tubing may well be shifted frequently by the patient, through movement, for comfort or by irritation; the tubing may become trapped with respect to other limbs and/or the surroundings of the patient.

As is known, medical lines may be provided for a number of different uses, such as the delivery of a drug, fluids and food (total parenteral nutrition, TPN). Other uses include measurement of central venous pressure and haemodialysis. TPN will drip through a needle or catheter placed within a vein for 10 to 12 hours, once a day or five times a week. TPN may include a combination of sugar and carbohydrates (for energy), proteins (for muscle strength), lipids (fat), electrolytes, and trace elements. Multiple lumens may be provided for the passage of different fluids.

FIG. 2 shows a first device 20 in accordance with the invention. The device comprises an arcuate member 22 and a support member 23. The arcuate member, defining a channel 24 within which a medical line member can lie, in use. The channel spans from a first cuff or tube locating member 25, to a second cuff 26, along the arcuate member. In use, both first and second cuffs ensure that a medical line attached lies within the channel, without twisting or bending whereby to enable a tube to be urged from a first, input direction of flow to a second, output direction of flow. Conveniently, the first cuff can comprises an aperture s dimensioned to circumferentially grip the medical line, whereby the device is not liable to become detached from the line, with associated contamination issues. Medical lines typically comprise resilient plastics tubing and it has been found that the if the inside diameter of the aperture corresponds with the nominal tube diameter of the medical line, then the tube can be gently and resiliently be held by first cuff, whilst also allowing the position of the device to be moved relative to the medical line or tube. In this example, the device enables a medical line to be directed 180° to an input flow direction, without exceeding minimum bend angles for the medical line. It will be appreciated that the angle defined by the arc may be 120° or 150°, whereby a medical line may be at a distinct angle to the input tube flow direction so that tube to be bent, within the minimum bend angles. The present invention can be manufactured from a large range of materials, conveniently recognised medical grade thermo-plastics, such as ABS, nylon, polypropylene, styrene etc.

FIG. 3 shows the first embodiment from a different perspective view and clearly shows the first closed aperture 31 defined by the first cuff 25 and the second, open aperture or yoke 32 of the second cuff. Note the cuff 26 also clearly shows the channel 24, which is defined to correspond with standard diameter medical lines—also in correspondence with the minimum bend radius for medical lines of that diameter, as discussed above. Additionally, the cuff has the opening width as defined between the opposing members of the yoke, 33 and 34 narrower than the channel width, whereby to enable resilient retention of a medical line once in place. Note that the outside of the channel conveniently has a ribbed surface decoration, whereby to enable grip as between the hands of a health care professional and the device, although this is quite optional. The support member or bridge between the cuffs need not be of any distinct shape, but must serve to provide structural integrity to the arcuate member 22 and may be a simple planar member or may have spokes directed from the underside/inside curve 35.

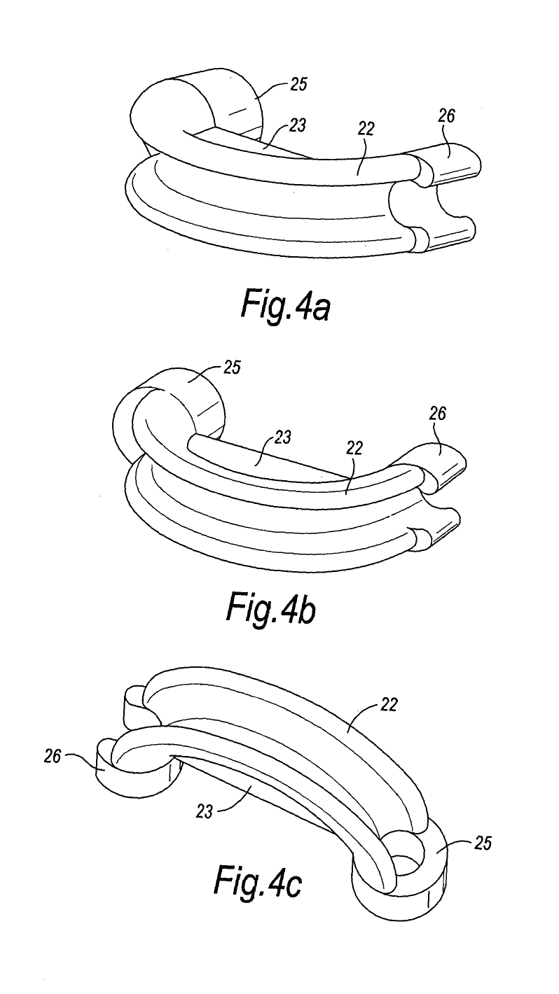

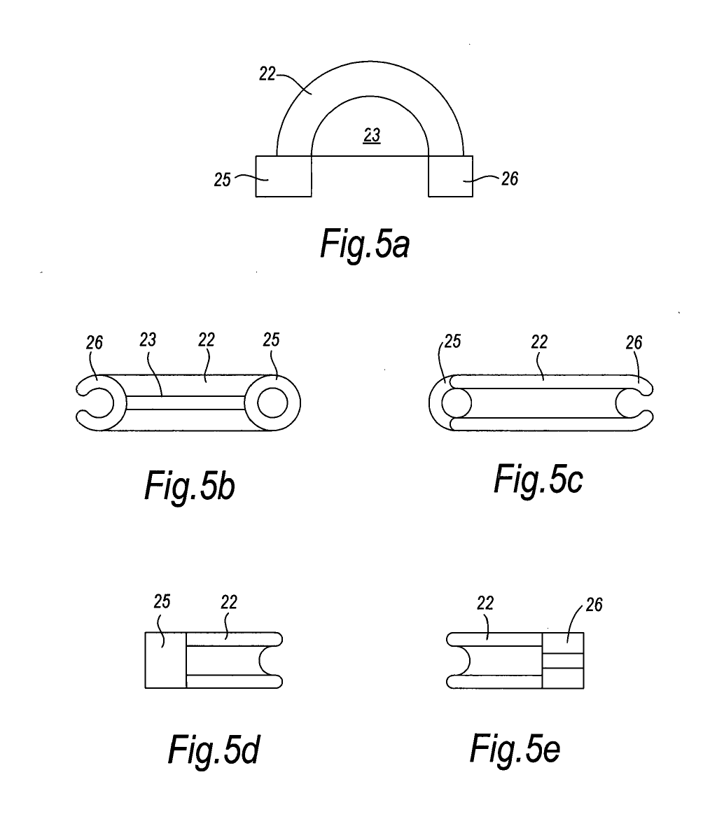

FIGS. 4a, 4b & 4c show alternative perspective views of the first embodiment. FIGS. 5a-5e show side, plan view of underneath of the channel, plan view of the channel and plan views of the first and second end. As detailed above, standard gauge sizes are employed in the io medical line industry; the width and radii of the arcuate channels are determined by the specific choice of medical line.

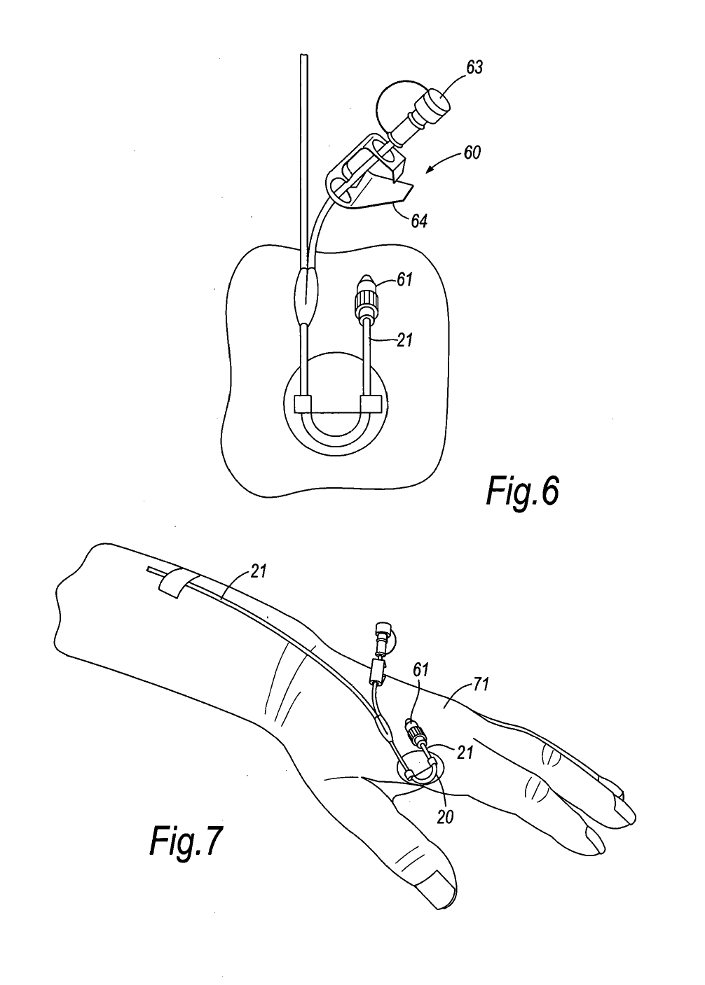

FIG. 6 shows how an IV kit 60 can be affixed to a body part wherein the direction of flow of a liquid to/from a vein can be reversed, through, conveniently 180° (or another selected angle defined during the manufacture of the device) of manufacture. In this figure, the cannula element 61 connects with a tube 21; the present invention provides a uniform radius about which the direction of the tube is changed by 180°, without fear of kink or blockage. The distal end of the tube 21 can be connected to a drip supply, for example a saline drip. Additionally, a tube combiner 62 is provided, connected to a port 63, for the delivery of a drug, for example, from a piggy-back drip supply. FIG. 7 shows a photograph of the present invention in use.

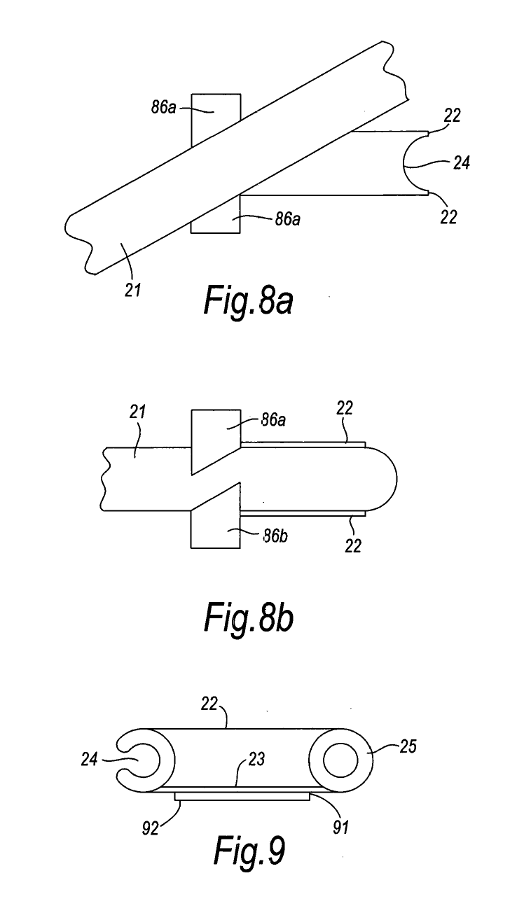

The present invention conveniently and safely provides a sterile, stable, efficient way to alter the direction of a medical line without fear of interrupting flow by kinking or otherwise. Whilst the invention has been shown as being attached to the medical line by way of the line going through a closed aperture 25, this is not the only means of securement: a 45° slit may be provided relative to the circumference of the first cuff, whereby a line may be placed through the aperture and subsequently rotated within the cuff so that it is resiliently received within the arcuate channel, as shown in FIG. 8. Other means of attachment/securement are possible; for example, the line may be secured by a grip or yoke defining an aperture, the aperture cuff has the opening width as defined between the opposing members of the yoke being less than the width of the medical line.

FIG. 9 shows a still further embodiment, where the support member 23, rather than being placed centrally with respect to the annular channel lies in a plane with one edge coterminous with the annular channel 22 and an adhesive skin contacting patch member 91, provided with a protective io cover 92. Once the protective cover has been removed, then the adhesive enables the device to be attached to the skin of a patient, whereby to enable secure placement and correct directivity of the medical line for a period of time.

The patch member 91 desirably has a sufficient thickness and tackiness to secure the device and thus the medical line in order to inhibit relative movement between the medical line and the patient. The adhesive can be applied to the securement system in any of a variety of conventional ways, such as by transfer technology (e.g. thin film transfer tape) or by sputtering. For example a transfer adhesive available from Minnesota Mining and Manufacturing Company (3M), could be employed. Preferably, the patch member 81 comprises a laminate structure with an upper foam layer (e.g., closed-cell polyethylene foam), and a bottom adhesive layer. The adhesive is, conveniently, a medical-grade adhesive and can be either diaphoretic or non-diaphoretic, depending upon the particular application. The patch member 91 desirably is formed from a polyethylene foam tape (1-2 mm thick) with an acrylic adhesive, 40 to 120 grams/square meter thick.

The removable paper or plastic backing or liner (not shown) suitably covers an underside adhesive surface before use. This release layer preferably resists tearing and is divided into a plurality of pieces to ease attachment of the pad to a patient's skin. In the illustrated embodiment, the backing is a polycoated, siliconized paper. The backing desirably is conveniently split along a centre line of the flexible contact patch member in order to expose only half of the adhesive bottom surface at one time. The backing liner also advantageously extends beyond at least one edge of the contact patch member, to facilitate removal of the backing liner from the adhesive layer.

One skilled in the art may also find additional applications for the devices and devices disclosed herein. Thus, the illustrations and descriptions of to securement devices disclosed herein in connection with a catheter are merely exemplary of one possible application of the device, in relation to a human patient, although it will be appreciated that there are numerous veterinary applications possible. Specific details of the apparatus can be altered: for example, the distal end of the tail can be made with a smoother end; the tail can be of several lengths; indeed, once in use, to would be possible to shorten the length of the tail, although this would reduce ease of removal and adjustment and care would need to be taken to ensure other, adjacent lines are not cut.

1. A medical line device comprising a first cuff, a second cuff and an arcuate channel member, the arcuate channel subtending an angular range, the first and second cuffs being associated with first and second ends of the arcuate channel;
Wherein the first cuff is shaped to retain a first portion of medical line when placed therebetween, the medical line being connectable with respect to one of a patient or medical supply;
Wherein the second cuff is shaped to retain a second portion of said medical line, with an intermediate portion of medical line between the cuffs lying within the arcuate channel, the medical line being connectable with respect to one of a medical supply or patient;
Whereby the medical line at the second cuff is directed in a different direction, as determined by the angular spread of the arcuate channel.

2. A medical line device according to claim 1, wherein the angular spread of the arcuate channel is between 45° and 180°, whereby the axial direction of the medical line is adjustable from 45° to 180°.

3. A medical line device according to claim 1, wherein the first cuff comprises a closed aperture.

4. A medical line device according to claim 1, wherein the first cuff comprises an open yoke.

5. A medical line device according to claim 1, wherein the first cuff comprises a generally ring-like member, wherein a slit defined in the ring, angled with respect to the circumference and the arcuate channel whereby a medical line can be placed through the slit and twisted such that it can lie coaxial with the channel and be resiliently retained within the cuff.

6. A medical line device according to claim 1, wherein the second cuff comprises a closed aperture.

7. A medical line device according to claim 1, wherein the second cuff comprises an open yoke.

8. A medical line device according to claim 1, wherein the second cuff comprises a generally ring-like member, wherein a slit defined in the ring, angled with respect to the circumference and the arcuate channel whereby a medical line can be placed through the slit and twisted such that it can lie coaxial with the channel and be resiliently retained within the cuff.

9. A medical line device according to claim 1, wherein the body of the arcuate channel has a textured surface to provide grip.

10. A medical line device according to claim 1, wherein the body of the support member extends in a plane such that it is generally coplanar with an exterior edge of the arcuate channel, and is provided with an adhesive patch whereby it can operably adhere to a patient.

11. A medical line device according to claim 1 wherein the body of the support member extends in a plane such that it is generally coplanar with an exterior edge of the arcuate channel, and is provided with an adhesive patch whereby it can operably adhere to a patient; wherein the adhesive patch comprises a laminate structure with an upper foam layer, and a bottom adhesive layer.

12. An intravenous medical line comprising a medical line, a cannula, a drip chamber, a fluid supply, and a medical line device according to claim 1.

13. A medical line device substantially as described herein with reference to any one or more of the figures as shown in the drawing sheets or the use of a medical line device to secure a medical line substantially as described herein with reference to any one or more of the figures as shown in the drawing sheets.

14. (canceled)

15. A method of preparing a medical line retainer device, said retainer device comprising a first cuff, a second cuff and an arcuate channel member, the arcuate channel subtending an angular spread, the first and second cuffs being associated with first and second ends of the arcuate channel;
the method comprising the step of placing a medical line with respect to a first cuff of the retainer device, so as to retain the medical line therewith, placing the medical line with respect to a second cuff of the retainer device so, as to retain the medical line therewith, adjusting the medical line with respect to said first and second cuffs;
the medical line being connectable and disconnectable with respect to one of an intravenous connection associated with a patient and a medical supply;
whereby the medical line at the second cuff is directed in a different direction with respect to the first cuff, as determined by the angular spread of the arcuate channel.
